(19) United States
(12) Patent Application Publication
(10) Pub. No.: US 2024/0248084 A1
(43) Pub. Date: Jul. 25, 2024

(54) METHOD FOR USING BIOTIN-COATED SOLID SUPPORT IN BIOCHEMICAL ASSAYS BASED ON INTERFEROMETRY

(71) Applicant: ACCESS MEDICAL SYSTEMS, LTD., Palo Alto, CA (US)

(72) Inventors: Robert F. Zuk, Menlo Park, CA (US); Pu Li, Los Altos, CA (US); Vasundhra Bahl, Mountain View, CA (US); Samuel Yang, San Jose, CA (US)

(21) Appl. No.: 18/437,032

(22) Filed: Feb. 8, 2024

Related U.S. Application Data
(63) Continuation of application No. PCT/US2022/074665, filed on Aug. 8, 2022.
(60) Provisional application No. 63/231,676, filed on Aug. 10, 2021.

(51) Int. Cl. G01N 33/557; G01N 33/543

(52) U.S. Cl. CPC G01N 33/557; G01N 33/54306

(57) ABSTRACT

The present invention is directed to biochemical assay methods, which uses a biotin-immobilized solid support surface for quantitating an analyte or measuring kinetic binding in different samples, from about 3 to 20 times, while maintaining acceptable assay performance. The methods use a streptavidin capture solution comprising streptavidin or preferably streptavidin polymers. The biotin-immobilized solid support surface is regenerated after each cycle of reactions by contacting the surface in an acidic solution having pH about 1-4, followed by DMSO. The regeneration step removes the bound immunocomplex and leaves the biotin on the surface.

### CROSS-REFERENCE TO RELATED APPLICATION(S)

This application is a continuation of PCT/US2022/074665, filed Aug. 8, 2022; which claims the benefit of U.S. Provisional Application No. 63/231,676, filed Aug. 10, 2021. The contents of the above-identified applications are incorporated herein by reference in their entirety.

### TECHNICAL FIELD

The present invention related to a biochemical assay based on interferometry. The method uses a biotin-coated solid support and a streptavidin capture solution comprising streptavidin monomer or streptavidin polymer for biochemical assays based on interferometry. The biotin-coated solid support can be regenerated by contacting it with an acidic solution (pH about 1-4) and DMSO, after the completion of each cycle of reaction. The method can re-use the biotin-coated solid support from about 2 to 15 times in the assays.

### BACKGROUND

Label free detection methods such as bio-layer interferometry (BLI) and surface plasmon resonance (SPR) have become standard methods in the study of receptor/ligand binding in biomedical research and in the development of therapeutics. Throughout the healthcare industry, including research applications and drug development, cost containment is a major issue. The expense of label free sensors limits their application, and consequently, limits the potential contributions of label free sensors in research and development.

Typical approaches to reducing the cost of immunoassays or assays employing a binding pair entail minimizing manufacturing expenses for materials, labor, and facilities overhead.

Any method to recycle immune reagents typically centers upon disassociating the immune complex with a denaturing agent such as an acidic/basic pH solution, organic solvents, chaotropic agents, etc. However, the denaturation step often changes the antibody charge, hydration, hydrogen bonding and tertiary structure where it no longer binds to antigen. Exposing the antibody back to the initial binding conditions close to physiologic pH and ionic strength, is hoped to restore original binding activity, however, few antibodies can tolerate repeated exposures to denaturation conditions without adversely impacting some aspect of their binding properties and consequently, assay performance.

There is a need for reducing the cost of immunoassays while maintaining the assay performance at the same time.

### BRIEF DESCRIPTION OF THE DRAWINGS

FIG. 1A depicts a biosensor interferometer that includes a light source, a detector, a waveguide, and an optical assembly (also referred to as a “probe”).

FIG. 1B depicts an example of a conventional probe.

FIG. 2 depicts another configuration of a probe.

FIGS. 3A-3B illustrate the principles of detection in a thin-film interferometer.

FIG. 4 illustrates the first embodiment of the invention for biotinylated analyte (biotin-CRP) quantitation. CRP (C-reactive protein) is used for illustration as the analyte.

FIG. 5 illustrates the second embodiment of the invention for analyte (antibody) quantitation. Anti-CRP antibody is used for illustration as the analyte.

FIG. 6 illustrates the third embodiment of the invention for antigen-antibody association and dissociation kinetics.

FIG. 7 shows the column elution profile of the streptavidin capture reagent and positions of the molecular weight calibrators run under identical conditions.

FIG. 8 shows anti-CRP antibody quantitation by an BLI method of the present invention.

FIG. 9 shows the representative BLI binding curves for one cycle of SA-CR, B-CRP, and anti-CRP at 0, 10, 30, 100 nM (association/disassociation).

FIG. 10 illustrates the consistency of the SA-CR and B-CRP BLI signals and derived KD of anti-CRP binding through 10 regeneration cycles.

FIG. 11 shows the BLI binding curves for SA-CR, B-PD-L1 and anti-PD-L1 at 0, 10, 30, 100 nM (association/disassociation).

FIG. 12 illustrates the consistency of the SA-CR signals, B-PD-L1 BLI signals, and derived KD of anti PD-L1 binding through 10 regeneration cycles.

FIG. 13 presents the BLI binding curves for SA-CR, B-TNF-α and anti-TNF-α.

FIG. 14 illustrates the consistency of the SA-CR BLI binding signals through 40 regeneration cycles in anti-TNF-α assay.

FIG. 15 presents BLI signals of B-TNF-α through 40 regeneration cycles.

FIG. 16 presents KDs of anti-TNF-α derived from association and disassociation BLI curves were consistent through 40 regeneration cycles.

FIG. 17 presents the BLI binding curves for SA-CR, B-EGFR and Cetuximab (anti-EGFR) at 0, 10, 30, 100 nM (association/disassociation).

FIG. 18 illustrates the consistency of the SA-CR signals, biotinylated EGFR BLI signals, and derived KD of Cetuximab binding through 10 regeneration cycles.

FIG. 19 shows consistent B-PD-L1 signals and anti-PD-L1 KDs through 5 cycles, and consistent B-CRP signals and anti-CRP KDs through 5 cycles, after alternating two analyte proteins in different cycles.

### DETAILED DESCRIPTION

### Definitions

Terms used in the claims and specification are to be construed in accordance with their usual meaning as understood by one skilled in the art except and as defined as set forth below.

“About,” as used herein, refers to within +10% of the recited value.

An “analyte-binding” molecule, as used herein, refers to any molecule capable of participating in a specific binding reaction with an analyte molecule. Examples include but are not limited to, (i) antigen molecules, for use in detecting the presence of antibodies specific against that antigen; (ii) antibody molecules, for use in detecting the presence of antigens; (iii) protein molecules, for use in detecting the presence of a binding partner for that protein; (iv) ligands, for use in detecting the presence of a binding partner; or (v) single stranded nucleic acid molecules, for detecting the presence of nucleic acid binding molecules.

“Antibody affinity” describes the strength of an antibody binds to an antigen.

“Antibody avidity” describes the measure of overall or accumulated strength of an antigen-antibody complex. It is determined by three parameters: the binding affinity of the complex, the valency of the antibody, and the structural arrangement of the antigen and the antibody in the complex that can be the cause multiple points of interaction.

An “aspect ratio” of a shape refers to the ratio of its longer dimension to its shorter dimension.

A “binding molecule,” refers to a molecule that is capable to bind another molecule of interest.

“A binding pair,” as used herein, refers to two molecules that are attracted to each other and specifically bind to each other. Examples of binding pairs include, but not limited to, an antigen and an antibody against the antigen, a ligand and its receptor, complementary strands of nucleic acids, biotin and avidin, biotin and streptavidin, lectin and carbohydrates. Preferred binding pairs are biotin and streptavidin, biotin and avidin, fluorescein and anti-fluorescein, digioxigenin/anti-digioxigenin.

“Immobilized,” as used herein, refers to reagents being fixed to a solid surface. When a reagent is immobilized to a solid surface, it is either be non-covalently bound or covalently bound to the surface.

“A monolithic substrate,” as used herein, refers to a single piece of a solid material such as glass, quartz, or plastic that has one refractive index.

A “probe,” as used herein, refers to a monolithic substrate having as aspect ratio (length-to-width) of at least 2 to 1 with a thin-film layer coated on the sensing side. A probe has a distal end and a proximal end. The proximal end (also refers to probe tip in the application) has a sensing surface coated with a thin layer of analyte-binding molecules.

The present invention related to a biochemical assay based on interferometry. The method uses a biotin-immobilized solid support, which is further coated with a streptavidin monomer or streptavidin polymer. The solid support can be regenerated and re-used from about 2 to 15 times, while maintaining acceptable assay performance, in a bio-layer interferometry (BLI) assay for quantitation or for kinetics. The present invention re-uses test solid support and optionally re-uses reagents, which and saves the cost on a per test basis. The present invention provides a method to regenerate biotin-coated probes, which bind to streptavidin monomers or streptavidin polymers, for multiple binding pair analysis using the same probe. Streptavidin is a tetramer with a molecular weight of approximately 53 KD (K Daltons) consisting of four subunits; each submit has a molecular weight of approximately 13 KD and contains a single biotin-binding site. The probe is initially coated with biotin, followed by binding to a streptavidin capture reagent (SA-CR). SA-CR of the present invention comprises streptavidin monomers or streptavidin polymers. Streptavidin polymers suitable for the present invention have a molecular weight of at least about 120 KD or about 145 KD, or at least about 465 KD, or at least about 970 KD. Streptavidin monomers and streptavidin polymers have multiple binding sites and after binding to the biotin coated probe, it can capture a biotin labeled member of a binding pair. The probe is then transferred to a sample with the second member of the binding pair. After monitoring the binding of the binding pair on the probe surface, the probe is transferred to a set of regeneration reagents to remove the streptavidin and the biotin labeled binding member. The biotin coated solid support is unaffected by the regeneration reagents and retains its streptavidin binding activity. The probe is then exposed to the SA-CR for a fresh coating of streptavidin for another sequence of binding steps. The probe can be cycled at least 10 times with most binding pairs by this invention. Since a fresh coating of SA-CR is used at each cycle, assay performance is maintained.

Solid support suitable for the present invention includes a probe, a bead, a flat surface (e.g., a slide) in flow cells, etc. Suitable materials of the solid support include glass, quartz, plastic, nitrocellulose, and nylon. Preferred solid support for the present invention is a probe made of glass or quartz.

The present invention uses bio-layer interferometry (BLI) for detecting the binding of the second member of the binding pair to the probe surface. BLI detection is advantageous because labels are not required that could alter the binding between the binding pair.

### Biosensor Interferometer Systems

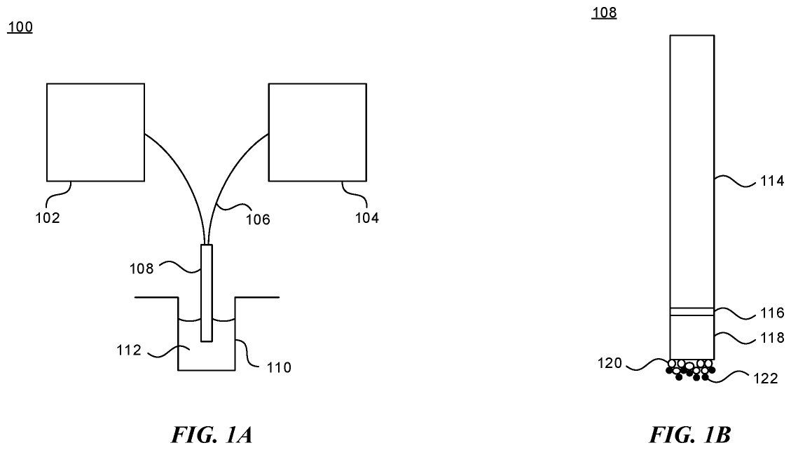

The present invention is suitable for several biosensor interferometer systems. FIGS. 1A-B illustrate one example of such a system in which a solid support is a probe. FIG. 1A depicts a biosensor interferometer 100 (or simply “interferometer”) that includes a light source 102, a detector 104, a waveguide 106, and an optical assembly 108 (also referred to as a “probe”). The probe 108 may be connected to the waveguide 106 via a coupling medium.

The light source 102 may emit white light that is guided toward the probe 108 by the waveguide 106. For example, the light source 102 may be a light-emitting diode (LED) that is configured to produce light over a range of at least 50 nanometers (nm), 100 nm, or 150 nm within a given spectrum (e.g., 400 nm or less to 700 nm or greater). Alternatively, the interferometer 100 may employ a plurality of light sources having different characteristic wavelengths, such as LEDs designed to emit light at different wavelengths in the visible range. The same function could be achieved by a single light source with suitable filters for directing light with different wavelengths onto the probe 108.

The detector 104 is preferably a spectrometer, such as an Ocean Optics USB4000, that is capable of recording the spectrum of interfering light received from the probe 108. Alternatively, if the light source 102 operates to direct different wavelengths onto the probe 108, then the detector 104 can be a simple photodetector capable of recording intensity at each wavelength. In another embodiment, the detector 104 can include multiple filters that permit detection of intensity at each of multiple wavelengths.

The waveguide 106 can be configured to transport light emitted by the light source 102 to the probe 108, and then transport light reflected by surfaces within the probe 108 to the detector 104. In some embodiments the waveguide 106 is a bundle of optical fibers (e.g., single-mode fiber optic cables), while in other embodiments the waveguide 106 is a multi-mode fiber optic cable.

As shown in FIG. 1B, the probe 108 includes a monolithic substrate 114, a thin-film layer (also referred to as an “interference layer”), and a biomolecular layer (also referred to as a “biolayer”) comprised of analyte molecules 122 that have bound to analyte-binding molecules 120. The monolithic substrate 114 is comprised of a transparent material through which light can travel. The interference layer is also comprised of a transparent material. When light is shone on the probe 108, the proximal surface of the interference layer may act as a first reflecting surface and the biolayer may act as a second reflecting surface. As further described below, light reflected by the first and second reflecting surfaces may form an interference pattern that can be monitored by the interferometer 100.

The interference layer normally includes multiple layers that are combined in such a manner to improve the detectability of the interference pattern. Here, for example, the interference layer is comprised of a tantalum pentoxide (Ta₂O₅) layer 116 and a silicon dioxide (SiO₂) layer 118. The tantalum pentoxide layer 116 may be thin (e.g., on the order of 10-40 nm) since its main purpose is to improve reflectivity at the proximal surface of the interference layer. Meanwhile, the silicon dioxide layer 118 may be comparatively thick (e.g., on the order of 650-900 nm) since its main purpose is to increase the distance between the first and second reflecting surfaces.

To perform a test, the probe 108 can be suspended in a microwell 110 (or simply “well”) that includes a sample 112. Analyte molecules 122 will bind to the analyte-binding molecules 120 along the distal end of the probe 108 over the course of the diagnostic test, and these binding events will result in an interference pattern that can be observed by the detector 104. The interferometer 100 can monitor the thickness of the biolayer formed along the distal end of the probe 108 by detecting shifts in a phase characteristic of the interference pattern.

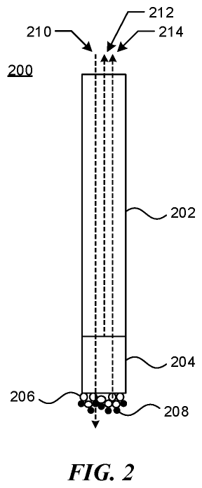

FIG. 2 illustrates another biosensor interferometer probe. The probe includes a monolithic substrate that has a first and a second surfaces arranged substantially parallel to one another at opposite ends of the monolithic substrate, an interference layer coated on the second surface of the monolithic substrate, and a layer of analyte-binding molecules coated on the interference layer. The interference layer will generally be comprised of magnesium fluoride (MgF2). A first interface between the monolithic substrate and the interference layer acts as a first reflecting surface when light is shone on the interferometric sensor, while a second interface between a biolayer formed by analyte molecules in a sample binding to the analyte-binding molecules and a solution containing the sample acts as a second reflecting surface when the light is shone on the probe. As described above, the thickness of the biolayer can be estimated based on the interference pattern of light reflected by the first and second reflecting surfaces.

The probe 200 includes an interference layer 204 that is secured along the distal end of a monolithic substrate 202. Analyte-binding molecules 206 can be deposited along the distal surface of the interference layer 204. Over the course of a biochemical test, a biolayer will form as analyte molecules 208 in a sample bind to the analyte-binding molecules 206.

As shown in FIG. 2, the monolithic substrate 202 has a proximal surface (also referred to as a “coupling side”) that can be coupled to, for example, a waveguide of an interferometer and a distal surface (also referred to as a “sensing side”) on which additional layers are deposited. Generally, the monolithic substrate 202 has a length of at least 3 millimeters (mm), 5 mm, 10 mm, or 15 mm. In a preferred embodiment, the aspect ratio (length-to-width) of the monolithic substrate 202 is at least 5 to 1. In such embodiments, the monolithic substrate 202 may be said to have a columnar form. The cross section of the monolithic substrate 202 may a circle, oval, square, rectangle, triangle, pentagon, etc. The monolithic substrate 202 preferably has a refractive index that is substantially higher than the refractive index of the interference layer 204, such that the proximal surface of the interference layer 204 effectively reflects light directed onto the probe 200. The preferred refractive index of the monolithic substrate may be higher than 1.5, 1.8, or 2.0. Accordingly, the monolithic substrate 202 may be comprised of a high-refractive-index material such as glass (refractive index of 2.0) rather than a low-refractive-index material such as quartz (refractive index of 1.46) or plastic (refractive index of 1.32-1.49).

The interference layer 204 is comprised of at least one transparent material that is coated on the distal surface of the monolithic substrate 202. These transparent material(s) are deposited on the distal surface of the monolithic substrate 202 in the form of thin films ranging in thickness from fractions of a nanometer (e.g., a monolayer) to several micrometers. The interference layer 204 may have a thickness of at least 500 nm, 700 nm, or 900 nm. An exemplary thickness is between 500-5,000 nm (and preferably 800-1,200 nm). Here, for example, the interference layer 204 has a thickness of approximately 900-1,000 nm, or 940 nm.

In contrast to conventional probes, the interference layer 204 has a substantially similar refractive index as the biolayer. This ensures that the reflection from the distal end of the probe 200 is predominantly due to the analyte molecules 208 rather than the interface between the interference layer 204 and the analyte-binding molecules 206. In some embodiments the interference layer 204 is comprised of magnesium fluoride (MgF₂), while in other embodiments the interference layer 204 is comprised of potassium fluoride (KF), lithium fluoride (LiF), sodium fluoride (NaF), lithium calcium aluminum fluoride (LiCaAlF₆), sodium aluminum fluoride (Na₃AlF₆), strontium fluoride (SrF₂), aluminum fluoride (AlF₃), sulphur hexafluoride (SF₆), etc. Magnesium fluoride has a refractive index of 1.38, which is substantially identical to the refractive index of the biolayer formed along the distal end of the probe 200. For comparison, the interference layer of conventional probes is normally comprised of silicon dioxide, and the refractive index of silicon dioxide is approximately 1.4-1.5 in the visible range. Because the interference layer 204 and biolayer have similar refractive indexes, light will experience minimal scattering as it travels from the interference layer 204 into the biolayer and then returns from the biolayer into the interference layer 204.

In one embodiment, the probe 200 includes an adhesion layer that is deposited along the distal surface of the interference layer 204 affixed to the monolithic substrate 202. The adhesion layer may be comprised of a material that promotes adhesion of the analyte-binding molecules 206. One example of such a material is silicon dioxide. The adhesion layer is generally very thin in comparison to the interference layer 204, so its impact on light traveling toward, or returning from, the biolayer will be minimal. For example, the adhesion layer 310 may have a thickness of approximately 3-10 nm, while the interference layer 304 may have a thickness of approximately 800-1,000 nm. The biolayer formed by the analyte-binding molecules 306 and analyte molecules 308 will normally have a thickness of several nm.

When light is shone on the probe 200, the proximal surface of the interference layer 204 may act as a first reflecting surface and the distal surface of the biolayer may act as a second reflecting surface. The presence, concentration, or binding rate of analyte molecules 208 to the probe 200 can be estimated based on the interference of beams of light reflected by these two reflecting surfaces. As analyte molecules 208 attach to (or detach from) the analyte-binding molecules 206, the distance between the first and second reflecting surfaces will change. Because the dimensions of all other components in the probe 200 remain the same, the interference pattern formed by the light reflected by the first and second reflecting surfaces is phase shifted in accordance with changes in biolayer thickness due to binding events.

In operation, an incident light signal 210 emitted by a light source is transported through the monolithic substrate 202 toward the biolayer. Within the probe 200, light will be reflected at the first reflecting surface resulting in a first reflected light signal 212. Light will also be reflected at the second reflecting surface resulting in a second reflected light signal 214. The second reflecting surface initially corresponds to the interface between the analyte-binding molecules 206 and the sample in which the probe 200 is immersed. As binding occurs during the biochemical test, the second reflecting surface becomes the interface between the analyte molecules 208 and the sample.

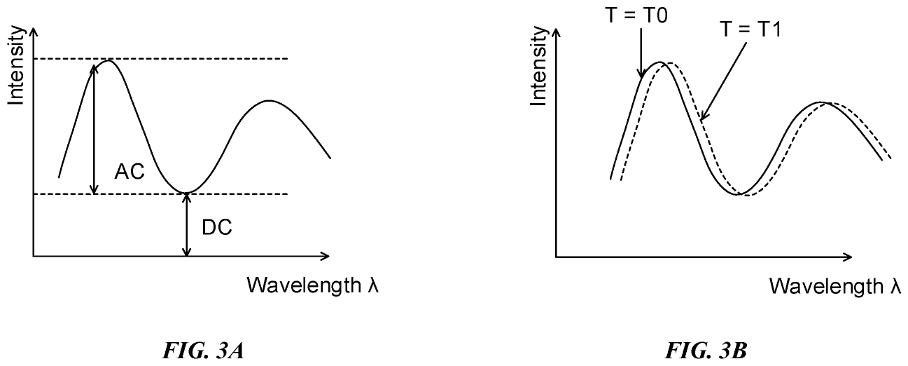

The first and second reflected light signals 212, 214 form a spectral interference pattern, as shown in FIG. 3A. When analyte molecules 208 bind to the analyte-binding molecules 206 on the distal surface of the interference layer 204, the optical path of the second reflected light signal 214 will lengthen. As a result, the spectral interference pattern shifts from T0 to T1 as shown in FIG. 3B. By measuring the phase shift continuously in real time, a kinetic binding curve can be plotted as the amount of shift versus the time. The association rate of an analyte molecule to an analyte-binding molecule immobilized on the distal surface of the interference layer 204 can be used to calculate analyte concentration in the sample. Hence, the measure of the phase shift is the detection principle of a thin-film interferometer.

### Assay Protocols Using Regenerated Biotin-Coated Solid Support and Streptavidin Capture Agent for Biochemical Assays

The present invention is directed to a biochemical assay. Optionally, the assay uses the same biotin-coated test probe for different samples, by regenerating the test probe with acidic treatment, followed by DMSO treatment.

### First Embodiment—Quantitation of Biotinylated Analyte

In a first embodiment, the method detects a biotinylated analyte. In some recombinant protein expression process, AviTag method incorporates a biotin tag at the end of the process. The present method can be used to quantify the expression level of the biotinylated protein.

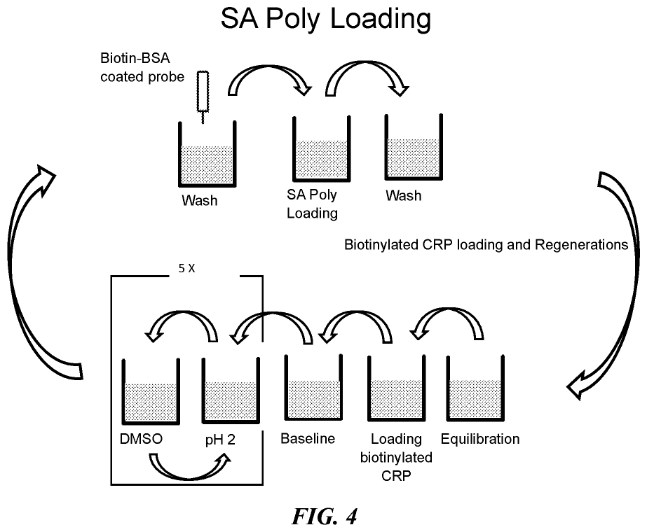

This embodiment is illustrated in FIG. 4 with CRP (C-reactive protein) as the analyte in the sample to be quantitated.

The method comprises the steps of: (a) obtaining a solid support having biotin immobilized on the surface of the solid support; (b) contacting the surface with a streptavidin solution comprising streptavidin or streptavidin polymers to bind the streptavidin to the surface; (c) contacting the surface with a wash solution; (d) contacting the surface with a first aqueous solution for a first period of time to determine a baseline interferometry pattern; (e) contacting the surface with a liquid sample having a biotinylated analyte for a second period of time to determine a second interferometry pattern of the immunocomplex formed at the surface; (f) determining the biotinylated analyte concentration in the sample by measuring the interferometry phase shift between the second interferometry pattern and the baseline interferometry pattern, and quantitating the phase shift against a calibration curve.

The method may further comprise the following steps after step (f) to regenerate the probe and re-use the probe: (g) contacting the surface with an acidic solution having pH about 1.0-4.0, followed by contacting the biotin-immobilized surface with DMSO to elute the immunocomplex from the surface, and leaving biotin immobilized on the surface; (h) contacting the biotin-immobilized surface with an aqueous solution having pH of 6.0-8.5, and (i) repeating steps (b)-(h) 3-15 times, except in step (c) of each cycle with a new liquid sample, whereby the analyte concentrations of multiple samples are determined.

In step (a) of the present method, one embodiment of the solid support a probe that has a small tip for binding an analyte is obtained. The tip has a smaller surface area with a diameter≤5 mm, preferably ≤2 mm or ≤1 mm. The small surface of the probe tip endows it with several advantages. In solid phase immunoassays, having a small surface area is advantageous because it has less non-specific binding and thus produces a lower background signal. Further, the reagent or sample carry over on the probe tip is extremely small due to the small surface area of the tip. This feature makes the probe tip easy to wash and results in negligible contamination in the wash solution since the wash solution has a larger volume. Another aspect of the small surface area of the probe tip is that it has small binding capacity. Consequently, when the probe tip is immersed in a reagent solution, the binding of the reagent does not consume a significant amount of the reagent. The reagent concentration is effectively unchanged. Negligible contamination of the wash solution and small consumption of the reagents enable the reagents and the wash solution to be re-used many times, for example, 3-10 times, 3-15 times, or 3-20 times.

Methods to immobilize biotin to the solid phase (the sensing surface of the probe tip) are common in immunochemistry and involve formation of covalent, hydrophobic or electrostatic bonds between the solid phase and a hapten. For example, biotin can be conjugated to a carrier protein and the biotin-protein is immobilized either by adsorption to the solid surface or by covalently binding to aminopropylsilane coated on the solid surface. Other methods for immobilization of biotin to a solid support may also be suitable.

In step (b) of the method, the surface of the solid support is contacted with a streptavidin solution comprising streptavidin or a streptavidin polymer. In a preferred embodiment, the streptavidin polymer has a molecular weight of at least 120 K or 145 K Dalton. The streptavidin or streptavidin polymer binds to biotin-coated solid support with 10¹⁵ M⁻¹ affinity.

In step (c), the solid support is washed by a wash solution having pH of 6.0-8.5 for a short time (e.g., 5 seconds to 5 minutes, 10 seconds to 2 minutes, or 30 seconds to 1 minute).

In step (d), the solid support surface is contacted with a first aqueous solution for a first period of time to determine a baseline interferometry pattern. The first aqueous solution is water or a buffer having pH between 6.0 to 8.5. Preferably, the aqueous solution contains 1-10 mM or 1-100 mM of phosphate buffer, tris buffer, citrate buffer or other buffer suitable for pH between 6.0-8.5.

In step (c), the solid support surface is contacted with a liquid sample having a biotinlyated analyte for a second period of time (e.g., 5 seconds to 5 minutes, 10 seconds to 2 minutes, or 30 seconds to 1 minute) to determine a second interferometry pattern of the immunocomplex formed at the surface for a second period of time.

In step (f), the biotinlyated analyte concentration in the sample is quantitated by determining the interferometry phase shift between the second interferometry pattern and the baseline interferometry pattern, and quantitating the wavelength phase shift against a calibration curve to determine the analyte concentration. In one embodiment, the calibration curve is the same for all cycles. In a preferred embodiment, the calibration curve is a cycle-specific calibration curve to adjust for any small change of signal caused by the regeneration process.

The phase shift can be monitored either kinetically or determined by the difference between starting time point (T0) and end time point (T1) (see FIG. 3B).

In step (g), the solid support surface is regenerated by employing a denaturing condition that dissociates the immune complexes bound to the solid support, and leaves biotin immobilized on the surface. In general, an acid or an acidic buffer having pH about 1 to about 4 is effective to regenerate the biotin-coated probe of the present invention. For example, hydrochloric acid, sulfuric acid, nitric acid, acetic acid can be used to regenerate the probe. The regeneration procedures can be one single acidic treatment, followed by neutralization. For example, a single pH 1-3, or pH 1.5-2.5 (e.g., pH 2) exposure ranging from 10 seconds to 2 minutes is effective. The regeneration procedures can also be a “pulse” regeneration step, where the solid support surface is exposed to 2-5 cycles (e.g. 3 cycles) of a short pH treatment (e.g., 10-20 seconds),

A dimethyl sulfoxide (DMSO) solution is used as a second elution agent after the acidic elution. In general, an aqueous solution (water or a buffer such as PBS) of DMSO is used with DMSO in an amount of 20-85%, 30-85%, or 40-80% by weight.

In step (h), after regeneration, the biotin-immobilized surface is contacted with an aqueous solution having pH of 6.0-8.5 (e.g., for at least 10-20 seconds) to neutralize the surface.

After regeneration and neutralization of the probe, steps of (b)-(h) are repeated with a different sample in a subsequent cycle, for 1-10, 1-20, 1-25, 3-20, 5-10, 5-20, 5-25, or 5-30 times, with the regenerated biotin-coated probe.

In one embodiment, when the solid support is a probe, the reaction can be accelerated by agitating or mixing the solution in the vessel that the probe is dipped in. For example, a flow such as a lateral flow or an orbital flow of the solution across the probe tip can be induced in one or more reaction vessels, including sample vessel, reagent vessel, wash vessels, and regeneration vessel, to accelerates the binding reactions, disassociation. For example, the reaction vessels can be mounted on an orbital shaker and the orbital shaker is rotated at a speed at least 50 rpm, preferably at least 200 rpm or at least 500 rpm, such as 50-200 or 500-1,500 rpm. Additionally, the probe tip can be moved up and down and perpendicular to the plane of the orbital flow, at a speed of 0.01 to 10 mm/second, in order to induce additional mixing of the solution above and below the probe tip.

### Second Embodiment—Quantitation of an Analyte

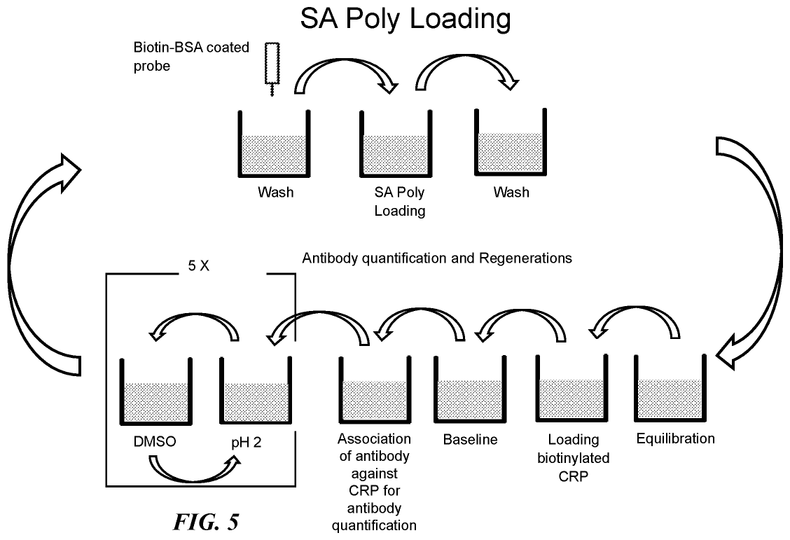

The second embodiment is illustrated in FIG. 5 with CRP (C-reactive protein) as the analyte in the sample to be quantitated.

The method comprises the steps of: (a) obtaining a solid support having biotin immobilized on the surface of the solid support; (b) contacting the surface with a streptavidin solution comprising streptavidin or streptavidin polymers to bind the streptavidin to the surface; (c) contacting the surface with a wash solution; (d) contacting the surface with a biotinylated first binding partner of a binding pair; (e) contacting the surface with a first aqueous solution for a first period of time to determine a baseline interferometry pattern; (f) contacting the surface with a liquid sample having an analyte for a second period of time to determine a second interferometry pattern of the immunocomplex formed at the surface, wherein the analyte is a second binding partner of the binding pair; (g) determining the analyte concentration in the sample by measuring the interferometry phase shift between the second interferometry pattern and the baseline interferometry pattern, and quantitating the phase shift against a calibration curve.

The method may further comprise the following steps after step (g) to regenerate the probe and re-use the probe: (h) contacting the surface with an acidic solution having pH about 1.0-4.0, followed by contacting the biotin-immobilized surface with DMSO to elute the immunocomplex from the surface, and leaving biotin immobilized on the surface; (i) contacting the biotin-immobilized surface with an aqueous wash solution having pH of 6.0-8.5, and (j) repeating steps (b)-(i) 3-15 times, except in step (f) of each cycle with a new liquid sample, whereby the analyte concentrations of multiple samples are determined.

In this method, a binding pair includes an antigen and an antibody against the antigen, a ligand and its receptor, complementary strands of nucleic acids, lectin and carbohydrates. Preferred binding pairs are antigen and antibody.

The details of each step are similar to those of a corresponding similar step, if any, described above in the first embodiment. For example, in step (b) of the method, the surface of the solid support is contacted with a streptavidin solution comprising streptavidin or a streptavidin polymer. In a preferred embodiment, the streptavidin polymer has a molecular weight of at least 120 kD or 145 K Dalton.

### Third Embodiment—Association and Dissociation Kinetics

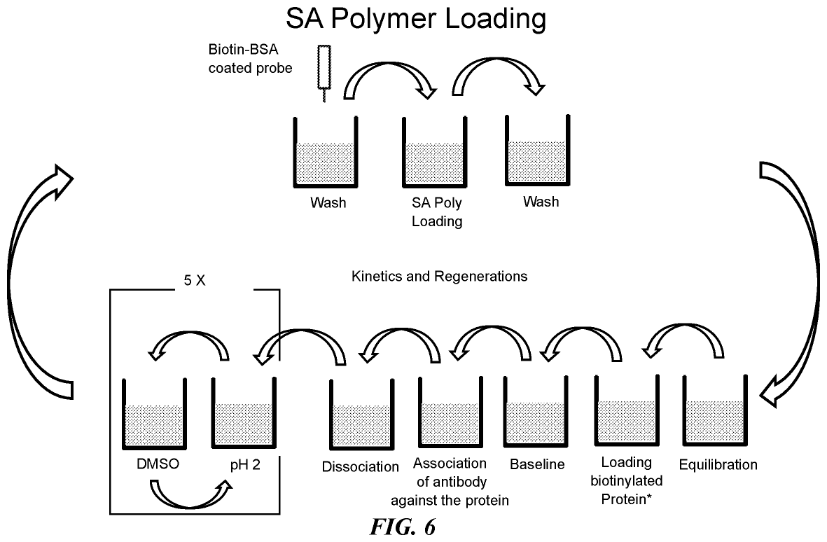

The third embodiment of the invention measures the association and dissociation kinetics of an antibody to an antigen in multiple samples each comprising an antibody. This embodiment is illustrated in FIG. 6.

The method is useful for measuring association rate and dissociation rate of an antibody to an antigen, and to determine the antibody affinity to the antigen. Overall determination of an antibody/antigen equilibrium binding constant is a ratio of the binding on-rate and off-rate. It is typically the dissociation rate that is the prime determinant of the equilibrium binding constant, consequently a dissociation rate measurement can be used to assess avidity of antibodies in clinical samples.

The method comprises the steps in the order of: (a) obtaining a solid support having biotin immobilized on the surface of the solid support; (b) contacting the surface with a streptavidin solution comprising streptavidin or streptavidin polymers to bind the streptavidin to the surface; (c) contacting the surface with a wash solution; (d) contacting the surface with a biotinylated antigen; (c) contacting the surface with a first aqueous solution for a first period of time to determine a baseline interferometry pattern; (f) contacting the surface with a liquid sample having an antibody against the antigen for a second period of time to determine a second interferometry pattern of the immunocomplex formed at the surface; (g) determining the interferometry phase shift between the second interferometry pattern and the baseline interferometry pattern to determine the association kinetics of the antibody to the antigen; (h) contacting the surface with a second aqueous solution for a second period of time to dissociate the antibody binding to the antigen and to determine a third interferometry pattern; (i) determining the interferometry phase shift between the third interferometry pattern and the second interferometry pattern and calculating the dissociation kinetics of the antibody and the antigen.

The method may further comprise the following steps after step (i) to regenerate the probe and re-use the probe: (j) contacting the surface with an acidic solution having pH about 1.0-4.0, followed by contacting the biotin-immobilized surface with DMSO to elute the immunocomplex from the surface, and leaving biotin immobilized on the surface; (k) contacting the biotin-immobilized surface with an aqueous solution having pH of 6.0-8.5, and (1) repeating steps (b)-(k) 3-15 times, except in step (f) of each cycle with a new liquid sample, whereby the dissociation kinetics of the antibody and the antigen in multiple samples are determined.

The details of each step are similar to those of a corresponding similar step, if any, described above in the first embodiment.

In step (b) of the method, the surface of the solid support is contacted with a streptavidin solution comprising streptavidin or a streptavidin polymer. In a preferred embodiment, the streptavidin polymer has a molecular weight of at least 100 K or 145 K Dalton.

Step (f) binds the antibody to the biotinylated antigen on the probe. Step (g) calculates the association rate (binding) of the antibody to the antigen. Step (h) dissociates the antigen from the antibody on the probe by contacting the surface in an aqueous solution that does not contain any antigen. Step (i) calculates the disassociation rate of the sample antibody to the sample antigen.

Biotin-streptavidin has a stronger affinity (femtomolar affinity) over the high affinity antigen/antibody binding, and thus streptavidin-CA bound to the biotin probe exhibits negligible dissociation during the dissociation of a test antibody from its antigen (step (h) above).

The biotherapeutic industry strives to develop high affinity antibodies. Recombinant antibodies with pico-molar and sub-pico molar affinities are now common and they are the focus in the development of new therapeutic antibodies. The present invention has advantages over other methods for determining the dissociation rate of a high affinity antibody, due to the negligible dissociation between SA-CR and biotin on the probe during the antibody dissociation step.

### Solid Support Surface Comprising Streptavidin Polymer

The present invention utilizes a solid support comprising a streptavidin polymer with a molecular weight of at least 120 KD or 145 KD bound to biotin-coated surface.

In one embodiment, the solid support is a probe having an aspect ratio of length to width of at least 5 to 1, the diameter of the probe tip surface is ≤5 mm.

The invention is illustrated further by the following examples that are not to be construed as limiting the invention in scope to the specific procedures described in them.

### EXAMPLES

### Example 1: Preparation of Biotin BSA

First, a solution of BSA (Jackson Immunoresearch, Catalog number: 001-000-162) at 20 mg/ml in 10 mM PBS, pH 7.4, and a solution of Biotin-NHS (Thermo Scientific) at 40 mg/ml in DMF was prepared. The Biotin NHS solution was added to the BSA solution at required volume (to keep 1 BSA to 30 Biotin) while vortexing slowly and was incubated for 1 hour at room temperature. After this, purification was done using a PD-10 column (GE Healthcare, Catalog number: 17-0851-01).

### Example 2. Preparation of Biotin-Coated Probes

Quartz probes, 1 mm diameter and 2 cm in length, were coated with aminopropylsilane (APS) using a chemical vapor deposition process (Yield Engineering Systems, 1224P) following manufacturer's protocols. Biotin regenerable probes were made by first washing the APS probe in ethanol and PBS for 10 minutes each and in water for 5 minutes. The probe was then washed in 10 mM sodium phosphate buffer pH 3.8 for 10 seconds under orbital shaking (500 rpm) and dipped into a solution of amine to amine cross linker BS3 (bis(sulfosuccinimidyl)suberate, Thermo Fisher, Catalog number: 21580) (1 mg/ml in 10 mM sodium phosphate buffer pH 3.8) for 2 minutes under 500 rpm. After that, the probes were washed in 10 mM sodium phosphate buffer pH 3.8 twice for 10 seconds each. This was followed by dipping into Biotin-BSA conjugate (1 mg/ml in 10 mM sodium phosphate buffer pH 3.8) under orbital shaking (1000 rpm) for 5 minutes. Finally, the probes were washed in PBS twice for 2 minutes and dipped into 15% sucrose before drying at 37° C. for 20 minutes. Table 1 shows the coating protocol.

TABLE 1

| | | | | | volume |
|---|---|---|---|---|---|
| Step type | Reagent | Conc | RPM | Time(sec) | (μL/well) |
| wash | Ethanol | | 1000 | 600 | 250 |
| wash | PBS | | 1000 | 600 | 250 |
| wash | Ultrapure water* | | 1000 | 600 | 250 |
| wash | 10 mM sodium | | 500 | 10 | 350 |
| | phosphate buffer | | | | |
| | pH 3.8 | | | | |
| Activation | BS3 1 mg/ml | 1 mg/ml | 500 | 120 | 200 |
| | in sodium | | | | |
| | phosphate buffer | | | | |
| | pH 3.8 | | | | |
| wash | 10 mM sodium | | 500 | 10 | 350 |
| | phosphate buffer | | | | |
| | pH 3.8 | | | | |
| wash | 10 mM sodium | | 500 | 10 | 350 |
| | phosphate buffer | | | | |
| | pH 3.8 | | | | |
| Coating | Biotin SC-BSA | 1 mg/ml | 1000 | 300 | 200 |
| | in sodium | | | | |
| | phosphate buffer | | | | |
| | pH 3.8 | | | | |
| wash/ | PBS | | 1000 | 120 | 250 |
| blocking | | | | | |
| wash/ | PBS | | 1000 | 120 | 250 |
| blocking | | | | | |
| Preservatives | 15% sucrose | 15% | 1000 | 60 | 250 |
| | in PBS | (w/v) | | | |

### Example 3. Preparing Streptavidin Polymer Reagent

Streptavidin polymer capture reagent (SA-CR) preparation involves making SA-SPDP and SA-SMCC, followed by mixing of the two. First, a solution of streptavidin (SA) (Prozyme, Catalog number: SA10) at 10 mg/ml in PBS was prepared and divided into two halves.

Preparation of SA-SPDP at a molar coupling ratio (MCR) of 1 SA to 7.5 SPDP: A solution of SPDP (succinimidyl 3-(2-pyridyldithio)propionate) (Thermo Scientific (Invitrogen)) at 10 mg/ml was prepared in DMF (Thermo Scientific). Required volume of SPDP solution (to keep the MCR at 7.5) was added to half of the SA solution and incubated at room temperature for 1 hour. Excess SPDP was then removed using a PD-10 column (GE Healthcare) and concentration of the elute was determined by OD 280. Next step involved the reduction of SA-SPDP by DTT (Thermo Scientific). DTT was prepared in PBS at a concentration of 38 mg/ml and added to SA-SPDP at a coupling ratio of 1 SA-SPDP to 100 DTT while gently vortexing. The mixture was incubated at room temperature for 1 hour. Excess DTT was then removed using PD-10 column. Concentration was measured by OD 280.

SA-SMCC at a coupling ratio of 1 SA to 7.5 SMCC: SMCC (Thermo Scientific) was prepared at 30 mg/ml in DMF and added to the remaining half of SA solution at required volume (to keep the MCR at 7.5). The mix was incubated at room temperature for 1 hour, after which PD-10 column was used to remove excessive SMCC. SA-SPDP-SH and SA-SMCC were mixed and incubated overnight at room temperature. Next day, N-Ethyl Maleimide (Thermo Scientific) at 32 mg/ml was added to the SA-CR at 0.011 ml per mg of SA and incubated at room temperature for 30 minutes to quench the reaction between reduced SPDP and SMCC.

Amicon Centriprep 50,000 MWCO (Millipore) was then used to concentrate the conjugate to desired volume after which the conjugate was loaded onto Sepharose CL-6B (SigmaAldrich). Sepharose CL-6B has a molecular size fraction range from 1×10⁴ Daltons to 4×10⁶ Daltons. The column was previously calibrated with several proteins of known molecular weights (Human IgM: 972 KD, beta galactosidase 465 KD, goat IgG 145 KD, streptavidin 60 KD).

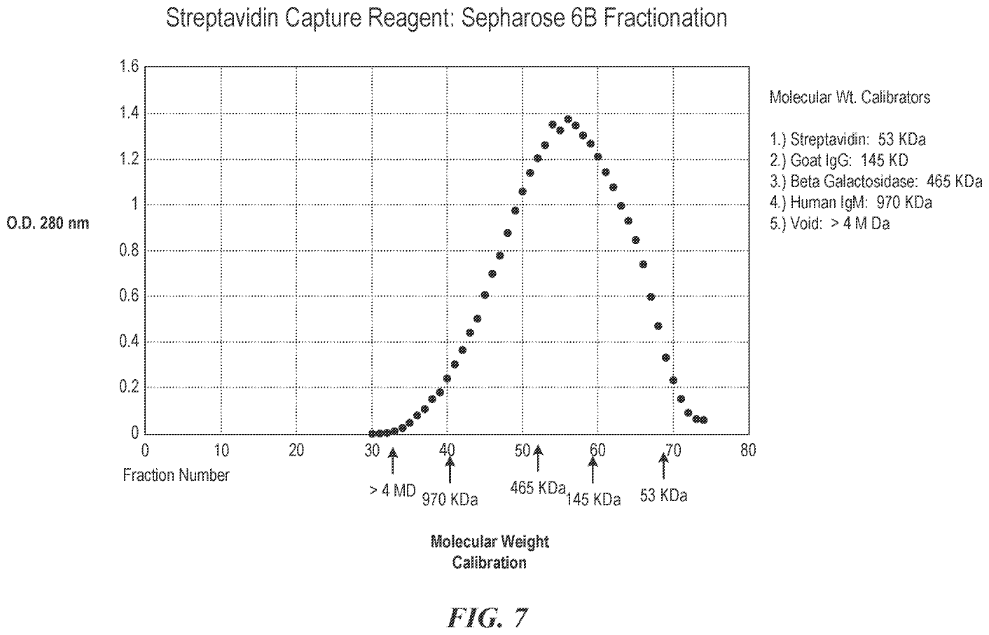

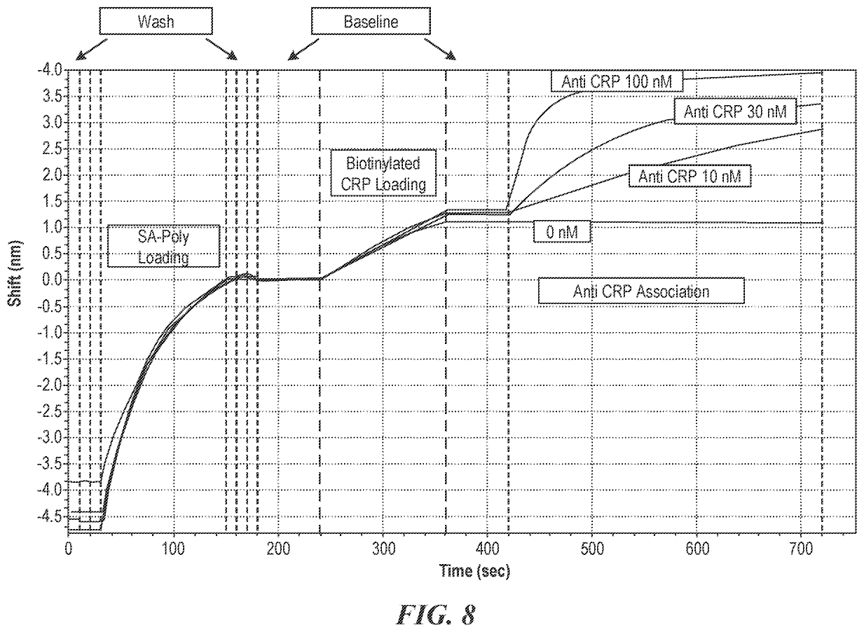

FIG. 7 shows the column elution profile of the SA-CR and positions of the molecular weight calibrators run under identical conditions. The SA preparation was polydisperse in its molecular size distribution. The calibration of the column enabled selection of fractions of the SA-CR of known molecular weights for subsequent evaluation in the regeneration assay.

### Example 4. Biotinylation of Human CRP

Human CRP (Sino Biological) was resuspended in PBS to make a 1 mg/mL solution. Then, EZ-Link™ NHS-PEG4-Biotin, No-Weigh™ Format (Thermo Scientific) was resuspended in 170 μL of DI water to make 20 mM biotin.

The biotin solution was mixed with CRP at 1:1 biotin:CRP molar coupling ratio (MCR) and incubated at room temperature for one hour. After one hour, excess biotin was removed by running through a Zeba™ Spin Desalting Columns, 7K MWCO (Thermo Scientific, Cat: 89882). The biotinylated protein was then collected in a clean microcentrifuge tube.

### Example 5. Biotinylation of Cetuximab

Cetuximab (R and D Systems) was resuspended in PBS to make a 1 mg/mL solution. Then, EZ-Link™ NHS-PEG4-Biotin, No-Weigh™ Format (Thermo Scientific) was resuspended in 170 μL of DI water to make 20 mM biotin.

The biotin solution was mixed with cetuximab at 1:1 biotin:Cetuximab molar coupling ratio (MCR) and incubated at room temperature for one hour. After one hour, excess biotin was removed by running through a Zeba™ Spin Desalting Columns, 7K MWCO (Thermo Scientific). The biotinylated protein was then collected in a clean microcentrifuge tube.

### Example 6. Biotinylation of Human TNF-α

Human TNF-α (Sino Biological) was resuspended in PBS to make a 1 mg/mL solution. Then, EZ-Link™ NHS-PEG4-Biotin, No-Weigh™ Format (Thermo Scientific, Cat: A39259) was resuspended in 170 μL of DI water to make 20 mM biotin.

The biotin solution was mixed with TNFα at 1:1 biotin:TNFα molar coupling ratio (MCR) and incubated at room temperature for one hour. After one hour, excess biotin was removed by running through a Zeba™ Spin Desalting Columns, 7K MWCO (Thermo Scientific). The biotinylated protein was then collected in a clean microcentrifuge tube.

### Example 7. BLI Measurements and Derivation of Antibody Dissociation Constants

Biolayer interferometry (BLI) measurements were taken using the Gator Prime (Gator Bio, Catalog number: 41-5000) or Gator Plus (Gator Bio, Catalog number: 41-5010) instruments according to the user's manual. Data analysis was done using the Gator software (version 1.7.2.1228). Both association and dissociation were used to obtain Kon, koff and kD values. 0 nM antibody curves were used as reference and subtracted from all other groups. The kD values were determined by using Global fit analysis to obtain the best fit lines for each biotinylated protein-antibody pair.

### Example 8. Effects of SA Polymers with Different Molecular Weights

The assay had two parts: SA-CR loading and biotinylated CRP loading.

The SA-CR loading protocol began by equilibrating the Biotin-BSA coated probes in PBS+0.2% BSA+0.02% tween 20 buffer for 5 minutes. They were then washed three times in PBS+0.2% BSA+0.02% tween 20 for 10 seconds each. Then, the probes were dipped into SA-CR of different molecular weights (different probe for each molecular weight) each at 50 μg/ml for 2 minutes under 1000 rpm. The molecular weights were as follows: 60 KD, 145 KD, 465 KD, and 970 KD, based on the Sepharose 6CL-B elution described in Example 3. This was followed by three washes in PBS+0.2% BSA+0.02% tween 20 for 10 seconds each.

For biotinylated CRP loading, probes were equilibrated in PBS+0.02% BSA+0.002% Tween 20+0.0005% Proclin for 30 seconds and were then loaded with biotinylated CRP at 3 μg/ml for 2 minutes under 1000 rpm orbital shaking. After that, the probes were dipped into PBS+0.02% BSA+0.002% tween 20+0.0005% Proclin for 60 sec. The probes were regenerated by alternatively dipping in to 10 mM Glycine buffer pH 2 and 100% DMSO 5 times for 5 seconds each. At the completion of Step 13 the probes were cycled back to Step 1 for another cycle. 5 regeneration cycles were performed. The assay protocol is shown in Table 2.

TABLE 2

| | Step | Reagent | Volume | rpm | time (sec) |
|---|---|---|---|---|---|
| Step 1 | Prewet | PBS + 0.2% BSA + 0.02% | 300 μl | 1000 | 300 |
| | | tween 20 + 0.005% Proclin | | | |
| Step 2 | Wash 1 | PBS + 0.2% BSA + 0.02% | 300 μl | 1000 | 10 |
| | | tween 20 + 0.005% Proclin | | | |
| Step 3 | Wash 2 | PBS + 0.2% BSA + 0.02% | 300 μl | 1000 | 10 |
| | | tween 20 + 0.005% Proclin | | | |
| Step 4 | Wash 3 | PBS + 0.2% BSA + 0.02% | 300 μl | 1000 | 10 |
| | | tween 20 + 0.005% Proclin | | | |
| Step 5 | SA-CR | SA-CR at 50 μg/ml in PBS + 0.2% | 300 μl | 1000 | 120 |
| | Loading | BSA + 0.02% tween 20 + 0.005% | | | |
| | | Proclin | | | |
| Step 6 | Wash 4 | PBS + 0.2% BSA + 0.02% | 300 μl | 1000 | 10 |
| | | tween 20 + 0.005% Proclin | | | |
| Step 7 | Wash 5 | PBS + 0.2% BSA + 0.02% | 300 μl | 1000 | 10 |
| | | tween 20 + 0.005% Proclin | | | |
| Step 8 | Wash 6 | PBS + 0.2% BSA + 0.02% | 300 μl | 1000 | 10 |
| | | tween 20 + 0.005% Proclin | | | |
| Step 9 | Baseline | PBS + 0.02% BSA + 0.002% | 250 μl | 1000 | 30 |
| | | tween 20 + 0.0005% Proclin | | | |
| Step 10 | Loading | B-CRP at 3 μg/ml in PBS + 0.02% | 250 μl | 1000 | 120 |
| | biotinylated | BSA + 0.002% tween 20 + 0.0005% | | | |
| | CRP | Proclin | | | |
| Step 11 | Signal | PBS + 0.02% BSA + 0.002% | 250 μl | 1000 | 60 |
| | | tween 20 + 0.0005% Proclin | | | |
| Step 12 | pH 2 | 10 mM Glycine pH 2 | 300 μl | 1000 | 5 |
| Step 13 | DMSO | 100% DMSO | 300 μl | 1000 | 5 |

Table 3-1 to 3-4 show results of BLI nm shifts of the SA-CR binding to the biotin probe. The wavelength (nm) shift progressively increased as the molecular weight increased from 60 KD to 960 KD. (2.8 nm to 4.05 nm).

TABLE 3-1
SA Monomer Mol wt 60,000 DA

| | Δ nm |
|---|---|
| Regeneration 1 | 3.242 |
| Regeneration 2 | 2.795 |
| Regeneration 3 | 2.687 |
| Regeneration 4 | 2.664 |
| Regeneration 5 | 2.671 |
| Avg ± SD | 2.812 ± 0.246 |

TABLE 3-2
SA Poly Mol wt 145,000 DA

| | Δ nm |
|---|---|
| Regeneration 1 | 3.726 |
| Regeneration 2 | 3.232 |
| Regeneration 3 | 3.180 |
| Regeneration 4 | 3.149 |
| Regeneration 5 | 3.133 |
| Avg ± SD | 3.284 ± 0.250 |

TABLE 3-3
SA Poly Mol wt 465,000 DA

| | Δ nm |
|---|---|
| Regeneration 1 | 3.871 |
| Regeneration 2 | 3.518 |
| Regeneration 3 | 3.455 |
| Regeneration 4 | 3.411 |
| Regeneration 5 | 3.390 |
| Avg ± SD | 3.529 ± 0.197 |

TABLE 3-4
SA Poly Mol wt 970,000 DA

| | Δ nm |
|---|---|
| Regeneration 1 | 4.393 |
| Regeneration 2 | 4.025 |
| Regeneration 3 | 3.963 |
| Regeneration 4 | 3.923 |
| Regeneration 5 | 3.948 |
| Avg ± SD | 4.050 ± 0.195 |

Tables 4-1 to 4-4 show results of BLI nm shifts of the biotin-CRP binding to the biotin-coated probe through streptavidin. The wavelength (nm) shift progressively increased as the molecular weight increased from 60 KD to 960 KD, (0.145 nm to 0.619 nm). These effects were consistent through 5 regeneration cycles.

TABLE 4-1
SA Monomer Mol wt 60,000 DA

| | Δ nm |
|---|---|
| Regeneration 1 | 0.117 |
| Regeneration 2 | 0.140 |
| Regeneration 3 | 0.153 |
| Regeneration 4 | 0.150 |
| Regeneration 5 | 0.166 |
| Avg ± SD | 0.145 ± 0.018 |

TABLE 4-2
SA Poly Mol wt 145,000 DA

| | Δ nm |
|---|---|
| Regeneration 1 | 0.272 |
| Regeneration 2 | 0.309 |
| Regeneration 3 | 0.334 |
| Regeneration 4 | 0.358 |
| Regeneration 5 | 0.376 |
| Avg ± SD | 0.330 ± 0.041 |

TABLE 4-3
SA Poly Mol wt 465,000 DA

| | Δ nm |
|---|---|
| Regeneration 1 | 0.361 |
| Regeneration 2 | 0.398 |
| Regeneration 3 | 0.434 |
| Regeneration 4 | 0.467 |
| Regeneration 5 | 0.499 |
| Avg ± SD | 0.432 |

TABLE 4-4
SA Poly Mol wt 970,000 DA

| | Δ nm |
|---|---|
| Regeneration 1 | 0.532 |
| Regeneration 2 | 0.579 |
| Regeneration 3 | 0.615 |
| Regeneration 4 | 0.663 |
| Regeneration 5 | 0.705 |
| Avg ± SD | 0.619 ± 0.068 |

The above results show a dependence of BLI binding signals on the molecular size of the SA-CR. As the SA-CR molecular weight increases from 60,000 Da to 970,000 Dalton, the binding of to the biotin-probe SA-CR increased about 1.4 fold, while the binding of Biotin-CRP unexpectedly increased even further about 4.3 fold. Maximizing the BLI binding signals of biotin-antigen is desirable for the subsequent affinity analysis with antibody. Further, streptavidin has an extremely high binding affinity KD, 10⁻¹⁵ M⁻¹, with 4 binding sites per streptavidin. SA-CR with molecular weights ranging from of 465 KDa to 970 KDa have 7 to 16 streptavidins in those conjugates, yielding an extremely high binding avidity. One might expect that the SA-CR would be difficult to reproducibly disassociate from probes with such high affinity and avidity; however, our data of Tables 3 and 4 show that regeneration of the probes exhibited no more than 11% CV (coefficient of variation) on BLI binding through 5 cycles.

Streptavidin polymer of molecular weight about 970,000 was used for the following Examples 9-13.

### Example 9: Regenerable CRP Assay for Kinetics Analysis

The assay had two parts: SA-CR loading and Kinetics. The assay protocol is summarized in Table 5.

TABLE 5

| | Step | Reagent | Volume | rpm | time (sec) |
|---|---|---|---|---|---|
| Step 1 | Prewet | PBS + 0.2% BSA + 0.02% | 300 μl | 1000 | 300 |
| | | tween 20 + 0.005% Proclin | | | |
| Step 2 | Wash 1 | PBS + 0.2% BSA + 0.02% | 300 μl | 1000 | 10 |
| | | tween 20 + 0.005% Proclin | | | |
| Step 3 | Wash 2 | PBS + 0.2% BSA + 0.02% | 300 μl | 1000 | 10 |
| | | tween 20 + 0.005% Proclin | | | |
| Step 4 | Wash 3 | PBS + 0.2% BSA + 0.02% | 300 μl | 1000 | 10 |
| | | tween 20 + 0.005% Proclin | | | |
| Step 5 | Sa-CR | SA-CR at 50 ug/mlin PBS + 0.2% | 300 μl | 1000 | 120 |
| | Loading | BSA + 0.02% tween 20 + 0.005% | | | |
| | | Proclin | | | |
| Step 6 | Wash 4 | PBS + 0.2% BSA + 0.02% | 300 μl | 1000 | 10 |
| | | tween 20 + 0.005% Proclin | | | |
| Step 7 | Wash 5 | PBS + 0.2% BSA + 0.02% | 300 μl | 1000 | 10 |
| | | tween 20 + 0.005% Proclin | | | |
| Step 8 | Wash 6 | PBS + 0.2% BSA + 0.02% | 300 μl | 1000 | 10 |
| | | tween 20 + 0.005% Proclin | | | |
| Step 9 | Equilibration | PBS + 0.02% BSA + 0.002% tween | 250 μl | 1000 | 30 |
| | | 20 + 0.0005% Proclin | | | |
| Step 10 | Loading | B-CRP at 3 μg/ml in PBS + 0.02% | 250 μl | 1000 | 120 |
| | biotinylated | BSA + 0.002% tween 20 + 0.0005% | | | |
| | CRP | Proclin | | | |
| Step 11 | Baseline | PBS + 0.02% BSA + 0.002% tween | 250 μl | 1000 | 60 |
| | | 20 + 0.0005% Proclin | | | |
| Step 12 | Association | PBS + 0.02% BSA + 0.002% tween | 250 μl | 1000 | 300 |
| | of anti-CRP | 20 + 0.0005% Proclin | | | |
| Step 13 | Dissociation | PBS + 0.02% BSA + 0.002% tween | 250 μl | 1000 | 600 |
| | | 20 + 0.0005% Proclin | | | |
| Step 14 | pH 2 | 10 mM Glycine pH 2 | 300 μl | 1000 | 5 |
| Step 15 | DMSO | 100% DMSO | 300 μl | 1000 | 5 |

The SA-CR loading protocol begins by equilibrating the Biotin-BSA coated probe in PBS+0.2% BSA+0.02% tween 20 buffer for 5 minutes. It was then washed three times in PBS+0.2% BSA+0.02% tween 20 for 10 seconds each. Then, the probe was dipped into SA-CR, 50 μg/ml, for 2 minutes under 1000 rpm. This was followed by three washes in PBS+0.2% BSA+0.02% Tween 20 for 10 seconds each.

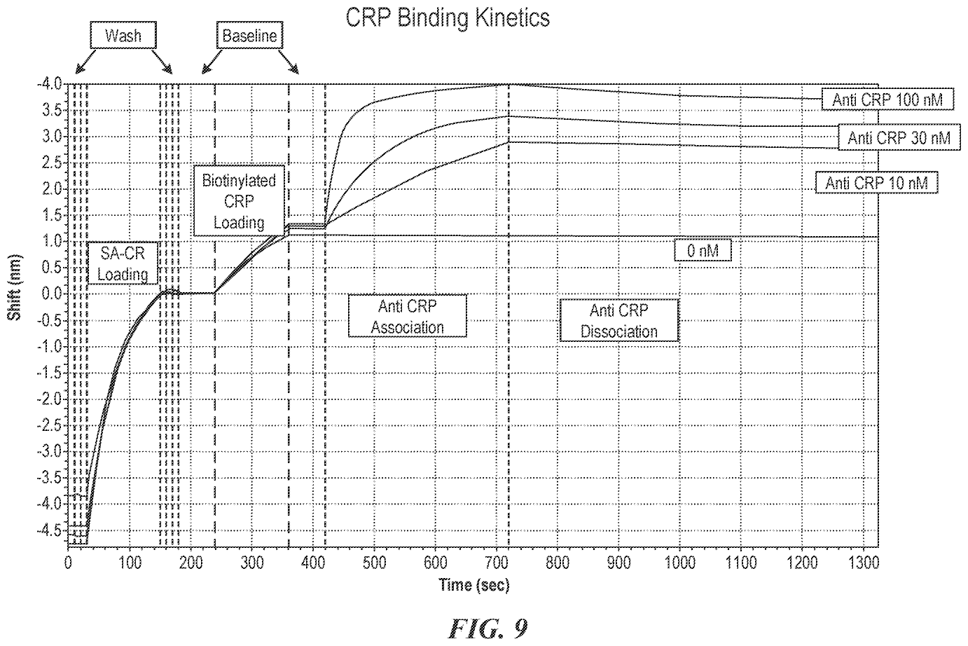

The probe was then equilibrated in PBS+0.02% BSA+0.002% tween 20+0.0005% Proclin for 30 seconds and was then transferred to a microwell containing biotinylated CRP at 3 μg/ml for 2 minutes under 1000 rpm orbital shaking. After that, the probe was dipped into PBS+0.02% BSA+0.002% tween 20+0.0005% Proclin for 60 sec and followed by transfer into microwells containing monoclonal anti CRP (HyTest. Catalog number: 4C28cc, clone 135cc) at various concentrations (0, 10, 30 and 100 nM) for 5 minutes to observe the real time association between CRP and anti-CRP. The probe was then dipped in PBS+0.02% BSA+0.002% tween 20+0.0005% Proclin for 10 minutes for the dissociation of anti-CRP from CRP. FIG. 9 shows the BLI binding curves for the SA-CR, B-CRP and anti-CRP. By analyzing the association curve and dissociation curve, the kinetic parameters such as kₒₙ, k_{off} and Kd were obtained. At the completion of the disassociation step, the probe was regenerated by dipping the probe into 10 mM Glycine buffer (pH 2), then into 100% DMSO 5 times for 5 seconds each to remove the SA-CR and CRP immune complex. The B-BSA remained on the probe during the regeneration. At the completion of step 15 of Table 5, where the SA-CR and CRP immune complex was removed, the probe was moved back to step 1 for subsequent cycles of SA-CR, B-CRP and anti CRP association/disassociation.

FIG. 9 shows the representative BLI binding curves for cycle of SA-CR, B-CRP, and anti-CRP at 0, 10, 30, 100 nM (association/disassociation).

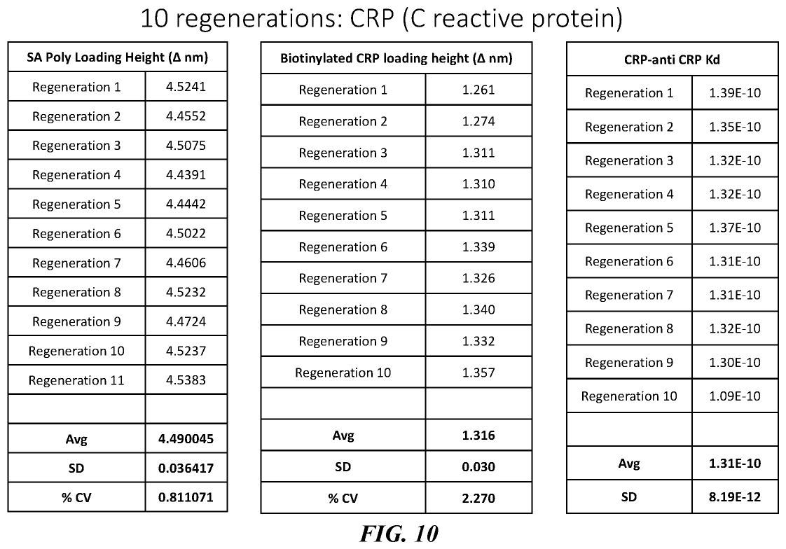

FIG. 10 illustrates the consistency of the SA-CR and B-CRP BLI signals and derived KD of anti CRP binding through 10 regeneration cycles.

KD affinity measurements are derived from several association binding curves and disassociation binding curves where software curve fitting algorithms generate an overall affinity constant. Consequently, the approach has many sources of variation, so KDs within a factor of 2 or 3 are deemed acceptable. The goal of therapeutic antibody development is to improve the KDs by a factor of at least 5 or 10-fold in order to expect an improved biological effect. In instances where the most accurate KD is needed, researches often run 3-4 replicate KD determinations, then calculate the average KD.

### Example 10: Regenerable PD-L1 Assay for Kinetics Analysis

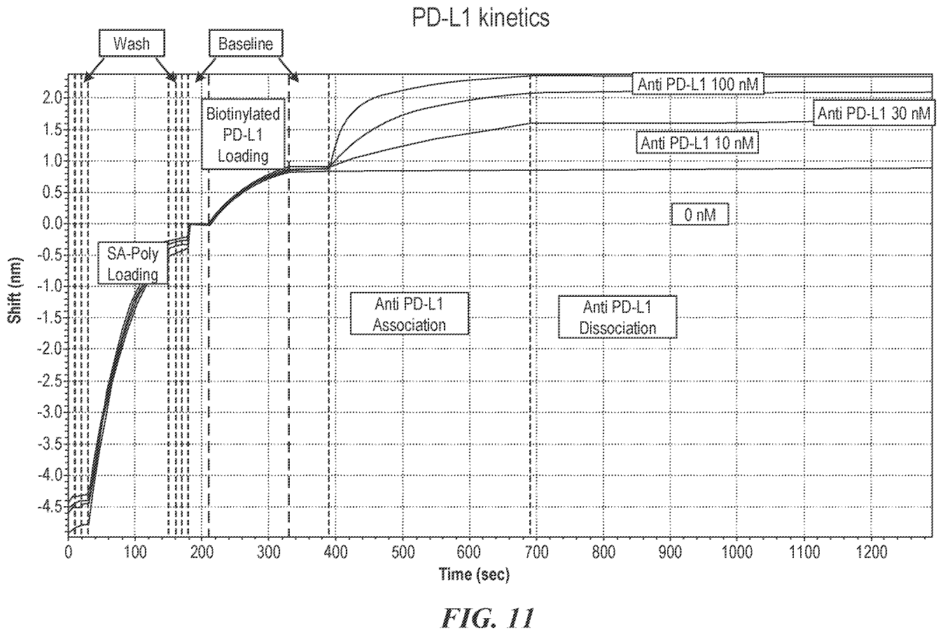

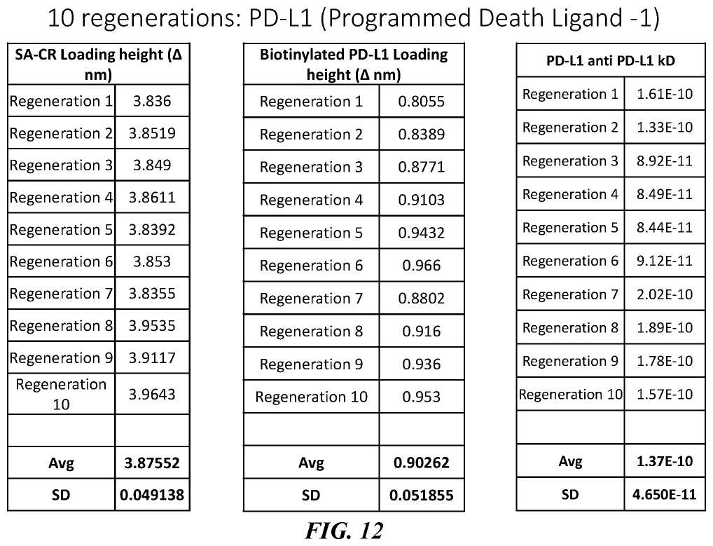

The regenerable assay for PD-L1 antibody followed the exact same protocol as Example 9, except the biotinylated protein was B-PD-L1 loaded at 50 μg/ml at Step 10, and the anti-PD-L1 (Sino Biological) was 0, 10, 30, or 100 nM at Step 12 for the antibody association to the PD-L1 on the probe. FIG. 11 presents the BLI binding curves for SA-CR, B-PD-L1 and anti-PD-L1. FIG. 12 illustrates the consistency of the SA-CR signals, B-PD-L1 BLI signals, and derived KD of anti PD-L1 binding through 10 regeneration cycles.

### Example 11: Regenerable TNF-Alpha Assay for Kinetics Analysis

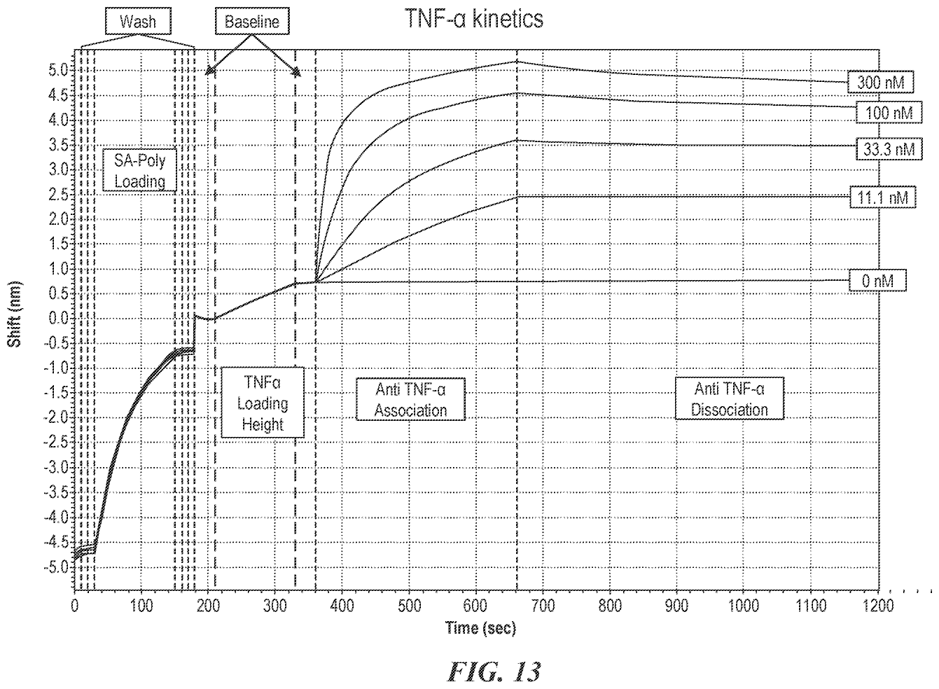

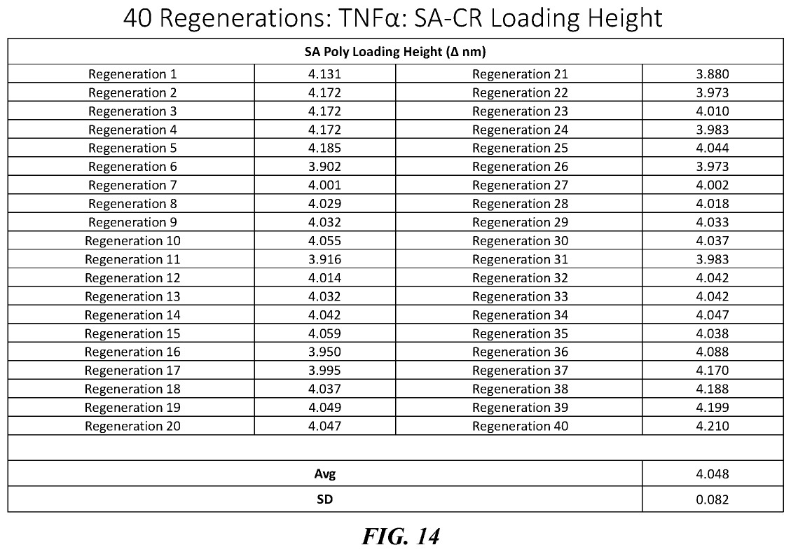

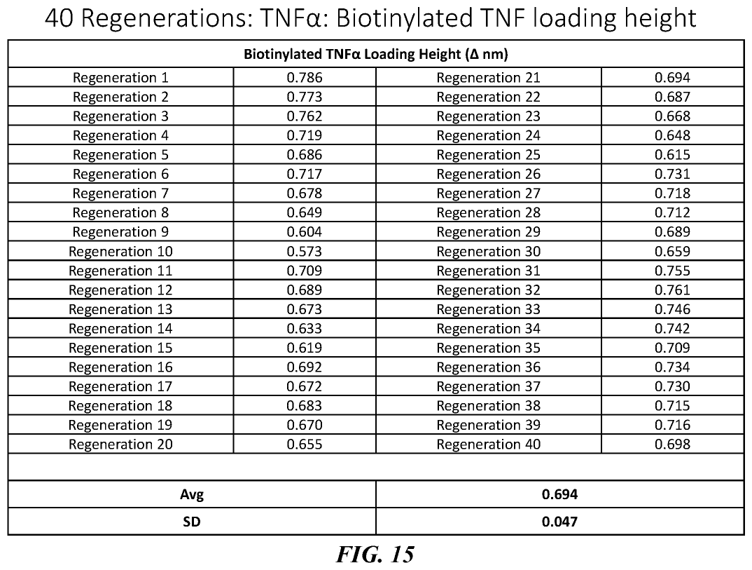

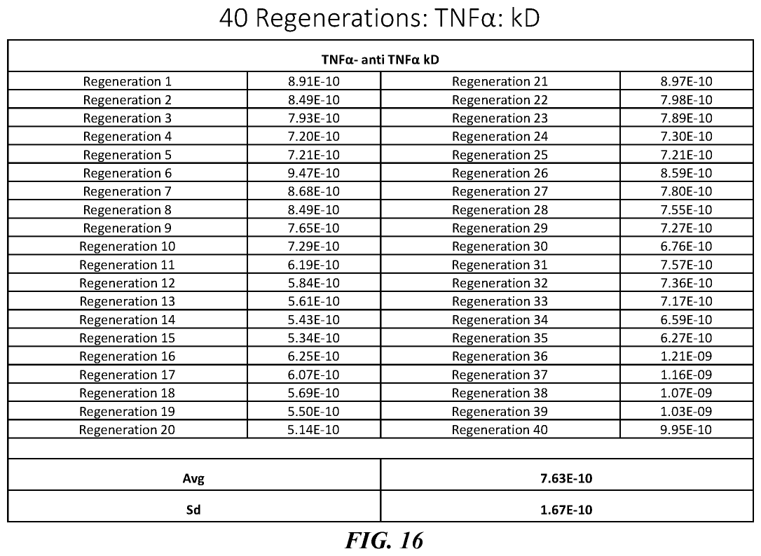

The regenerable assay for TNF-alpha antibody followed the exact same protocol as Example 9, except the biotinylated protein was B-PD-L1 loaded at 50 μg/ml at Step 10, and the rabbit polyclonal anti-TNF-α (Sino Biological) was 0, 11.1, 33.3, and 100 nM at Step 12 for the antibody association to the B-TNF-α pha on the probe. FIG. 13 presents the BLI binding curves for SA-CR, B-TNF-α and anti-TNF-α. FIG. 14 illustrates the consistency of the SA-CR BLI binding signals through 40 regeneration cycles. FIG. 15 presents BLI signals of B-TNF-α through 40 regeneration cycles. KDs derived from association and disassociation BLI curves from multiple anti-TNF-alpha concentrations (11,1, 33,3, 100, and 300 nM) were consistent through 40 regeneration cycles (FIG. 16).

### Example 12: Regenerable Cetuximab Assay for Kinetics Analysis

The regenerable assay for Cetuximab antibody (anti-EGFR) followed the exact same protocol as Example 9, except the biotinylated protein was B-EGFR loaded at 50 μg/ml at Step 10, and the Cetuximab (R & D System)) was 0, 10, 30, or 100 nM at Step 12 for the antibody association to the EGFR on the probe.

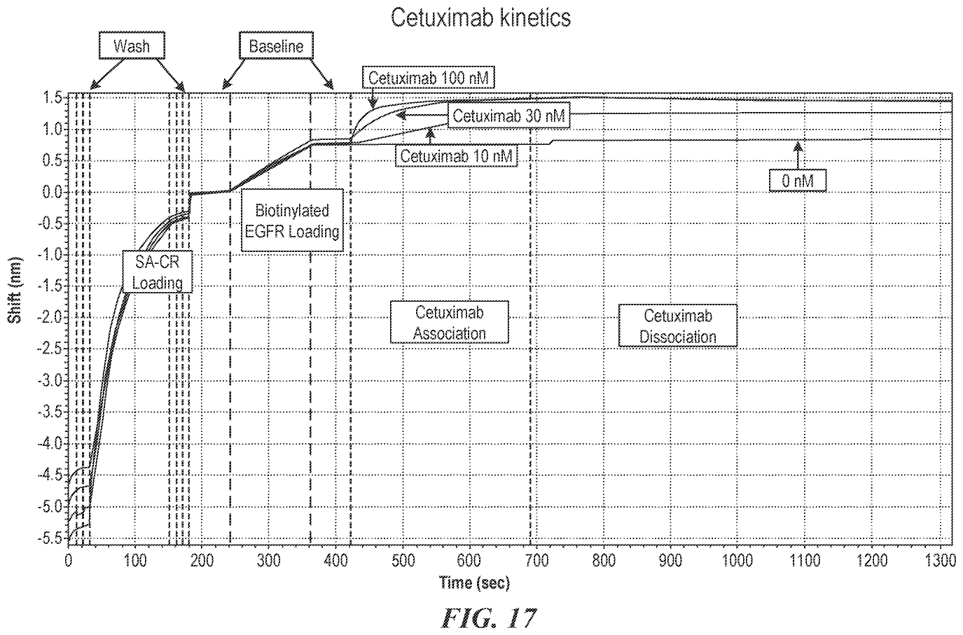

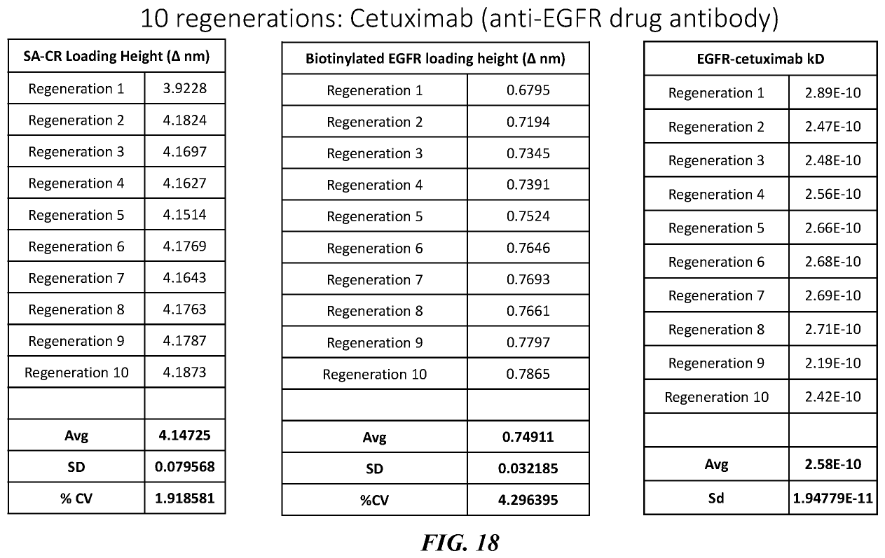

FIG. 17 presents the BLI binding curves for SA-CR, B-EGFR and Cetuximab. FIG. 18 illustrates the consistency of the SA-CR signals, EGFR BLI signals, and derived KD of Cetuximab binding through 10 regeneration cycles.

Examples 9-12 demonstrate the regeneration assay can be applied to several antigen/antibody pairs with at least 10 regeneration cycles with consistent results.

### Example 13: Regeneration Assay Alternating Between 2 Different Antigen/Antibody Pairs Using Same Biotin Probe

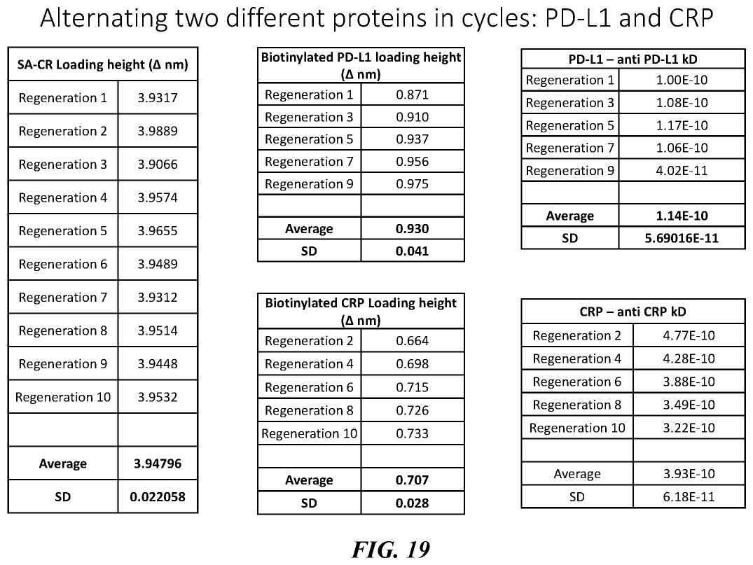

10 regeneration cycles were performed to derive KDs using the same biotin probe and SA-CR. For even numbered cycles, B-PD-L1 and anti-PD-L1 were used following the protocol as in Example 10; and for odd numbered cycles, B-CRP and anti-CRP were used following the protocol as in Example 9. FIG. 19 shows consistent B-PD-L1 signals and anti-PD-L1 KDs through 5 cycles, and consistent B-CRP signals and anti-CRP KDs through 5 cycles. The results indicate that there was negligible interference between two different analytes in the regeneration assay. The results show that the elution and regeneration steps in the invention appear efficient, which enables the use of different analyte binding pairs at various cycles without interference. The invention does not have to be restricted to a single binding pair with the same biotin probe and SA-CR reagent.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

I/We claim:

1. A method of detecting an analyte in multiple liquid samples comprising an analyte, comprising the steps of:
(a) obtaining a solid support having biotin immobilized on the surface of the solid support;
(b) contacting the surface with a streptavidin solution comprising streptavidin polymer having a molecular weight of at least about 145,000 Dalton to bind the streptavidin polymer to the surface;
(c) contacting the surface with a wash solution;
(d) contacting the surface with a biotinylated first binding partner of a binding pair;
(e) contacting the surface with a first aqueous solution for a first period of time to determine a baseline interferometry pattern;
(f) contacting the surface with a liquid sample having an analyte for a second period of time to determine a second interferometry pattern of the immunocomplex formed at the surface, wherein the analyte is a second binding partner of the binding pair; and
(g) determining the analyte concentration in the sample by measuring the interferometry phase shift between the second interferometry pattern and the baseline interferometry pattern, and quantitating the phase shift against a calibration curve.

2. The method of claim 1, further comprising the following steps after step (g):
(h) contacting the surface with an acidic solution having pH about 1.0-4.0, followed by contacting the biotin-immobilized surface with dimethyl sulfoxide (DMSO) to elute the immunocomplex from the surface, and leaving biotin immobilized on the surface;
(i) contacting the biotin-immobilized surface with an aqueous wash solution having pH of 6.0-8.5, and
(j) repeating steps (b)-(i) 3-15 times, except in step (f) of each cycle with a new liquid sample, whereby the analyte concentrations of multiple samples are determined.

3. The method of claim 1, wherein the first member of the binding pair is an antigen, and the second member of the binding pair is an antibody.

4. The method of claim 1, wherein the streptavidin polymer has a molecular weight of at least about 465,000 dalton.

5. The method of claim 1, wherein the streptavidin polymer has a molecular weight of at least about 970,000 dalton.

6. The method of claim 2, wherein the acidic solution has a pH of 1.5-2.5.

7. The method of claim 2, wherein DMSO is in a solution comprising 20-85% DMSO by weight.

8. A method of detecting a biotinylated analyte in multiple liquid samples comprising an analyte, comprising the steps of:
(a) obtaining a solid support having biotin immobilized on the surface of the solid support;
(b) contacting the surface with a streptavidin solution comprising streptavidin polymer having a molecular weight of at least about 145,000 Dalton to bind the streptavidin polymer to the surface;
(c) contacting the surface with a wash solution;
(d) contacting the surface with a first aqueous solution for a first period of time to determine a baseline interferometry pattern;
(e) contacting the surface with a liquid sample having a biotinlyated analyte for a second period of time to determine a second interferometry pattern of the immunocomplex formed at the surface; and
(f) determining the biotinylated analyte concentration in the sample by measuring the interferometry phase shift between the second interferometry pattern and the baseline interferometry pattern, and quantitating the phase shift against a calibration curve.

9. The method of claim 8, further comprising the following steps after step (f):
(g) contacting the surface with an acidic solution having pH about 1.0-4.0, followed by contacting the biotin-immobilized surface with DMSO to elute the immunocomplex from the surface, and leaving biotin immobilized on the surface;
(h) contacting the biotin-immobilized surface with an aqueous solution having pH of 6.0-8.5, and
(i) repeating steps (b)-(h) 3-15 times, except in step (e) of each cycle with a new liquid sample, whereby the analyte concentrations of multiple samples are determined.

10. The method of claim 8, wherein the streptavidin polymer has a molecular weight of at least about 465,000 dalton.

11. The method of claim 8, wherein the streptavidin polymer has a molecular weight of at least about 970,000 dalton.

12. The method of claim 9, wherein the acidic solution has a pH of 1.5-2.5.

13. The method of claim 9, wherein DMSO is in a solution comprising 20-85% DMSO by weight.

14. A method of measuring the association and dissociation kinetics of an antibody to an antigen in multiple samples each comprising an antibody, comprising the steps in the order of:
(a) obtaining a solid support having biotin immobilized on the surface of the solid support;
(b) contacting the surface with a streptavidin solution comprising streptavidin or streptavidin polymer having molecular weight of at least about 145,000 dalton to bind the streptavidin to the surface;
(c) contacting the surface with a wash solution;
(d) contacting the surface with a biotinylated antigen;
(e) contacting the surface with a first aqueous solution for a first period of time to determine a baseline interferometry pattern;
(f) contacting the surface with a liquid sample having an antibody against the antigen for a second period of time to determine a second interferometry pattern of the immunocomplex formed at the surface;
(g) determining the interferometry phase shift between the second interferometry pattern and the baseline interferometry pattern to determine the binding kinetics of the antibody to the antigen;
(h) contacting the surface with a second aqueous solution for a second period of time to determine a third interferometry pattern;
(i) determining the interferometry phase shift between the third interferometry pattern and the second interferometry pattern and calculating the dissociation kinetics of the antibody and the antigen;
(j) contacting the surface with an acidic solution having pH about 1.0-4.0, followed by contacting the biotin-immobilized surface with DMSO to elute the immunocomplex from the surface, and leaving biotin immobilized on the surface;
(k) contacting the biotin-immobilized surface with an aqueous solution having pH of 6.0-8.5, and
(l) repeating steps (b)-(k) 3-15 times, except in step (f) of each cycle with a new liquid sample, whereby the dissociation kinetics of the antibody and the antigen in multiple samples are determined.

15. The method of claim 14, wherein the streptavidin polymer has a molecular weight of at least about 465,000 dalton.

16. The method of claim 14, wherein the streptavidin polymer has a molecular weight of at least about 970,000 dalton.

17. The method of claim 14, wherein the acidic solution has a pH of 1.5-2.5.

18. The method of claim 14, wherein DMSO is in a solution comprising 20-85% DMSO by weight.
